(19) United States
(12) Patent Application Publication
(10) Pub. No.: US 2024/0298876 A1
(43) Pub. Date: Sep. 12, 2024

(54) Portable Medical Imaging System

(71) Applicant: Lazurite Holdings LLC, Cleveland, OH (US)

(72) Inventors: Laimis Kazimieras Belzinskas, Kirtland, OH (US); llya Malinskiy, Cleveland Heights, OH (US); Eugene Malinskiy, Mayfield Vilage, OH (US)

(21) Appl. No.: 18/551,467

(22) PCT Filed: Mar. 24, 2022
(86) PCT No.: PCT/US2022/021674

§ 371 Date: Sep. 20, 2023

Related U.S. Application Data
(60) Provisional application No. 63/218,620, filed on Jul. 6, 2021.
(60) Provisional application No. 63/165,935, filed on Mar. 25, 2021.

(51) Int. Cl. A61B 1/00; A61B 1/045; A61B 50/31; A61B 90/70

(52) U.S. Cl. CPC A61B 1/00048; A61B 1/00016; A61B 1/00034; A61B 1/00142; A61B 1/045; A61B 50/31; A61B 90/70; A61B 2090/701

(57) ABSTRACT

A portable imaging system includes a case having a first portion coupled to a second portion. The second portion includes an interior storage portion, a wireless imaging system that has a housing, a camera sensor disposed within the housing, and a light source. The portable imaging system further includes an imaging scope removably coupled to the wireless imaging system, a controller that is communicatively coupled to the wireless imaging system, and a display screen that is disposed on an interior surface of the first portion of the case. The display screen is communicatively coupled to the controller and the interior storage portion is configured to receive and store the wireless imaging system and the imaging scope.

### CROSS-REFERENCE TO RELATED APPLICATIONS

This application claims the benefit of U.S. Provisional Patent Application No. 63/165,935 filed Mar. 25, 2021, entitled “Portable Surgical Demonstration System” and U.S. Provisional Patent Application No. 63/218,620 filed Jul. 6, 2021, entitled “Portable Medical Imaging System”, each of which is incorporated by reference herein in its entirety.

### TECHNICAL FIELD

The present disclosure generally relates to a portable medical imaging system, and in some embodiments, a portable minimally invasive surgical visualization system.

### SUMMARY

One embodiment of the present disclosure provides a portable imaging system having a case having a first portion coupled to a second portion, the second portion including an interior storage portion, a wireless imaging system having a housing, a camera sensor disposed within the housing, and a light source, an imaging scope removably coupled to the wireless imaging system, a controller communicatively coupled to the wireless imaging system, and a display screen disposed on an interior surface of the first portion of the case, the display screen communicatively coupled to the controller. The interior storage portion may be configured to receive and store the wireless imaging system and the imaging scope.

In some embodiments, the portable imaging system further includes a sterilizing cover configured to be disposed over one or more of the imaging scope, the wireless imaging system, and a battery coupled to the wireless imaging system. A sterilizing device may be configured to sterilize an object received by the sterilizing cover.

In some embodiments, the case has an external shell comprised of one or more of a substantially hard plastic and rubber. The external shell may be comprised of a different material than a material of the interior storage portion. The material of the interior storage portion may be comprised of material configured to deform to attenuate force.

In some embodiments, the portable imaging system further includes an internal battery disposed within the case and coupled to one or more of the controller and the display screen, wherein the internal battery is configured to charge a battery coupled to the wireless imaging system. The wireless imaging system may further include a rechargeable battery. The camera sensor may transmit one or more images to the display screen via the controller. The controller may be wirelessly coupled to the wireless imaging system.

In some embodiments, the interior storage portion is comprised of material configured to dissipate force. The interior storage portion may include a first recess sized and shaped to secure the wireless imaging system and a second recess sized and shaped to secure the imaging scope, the first recess being different than the second recess.

In some embodiments, the case includes a locking mechanism to secure and lock the first portion to the second portion.

In some embodiments, the portable imaging system further includes a protective cover covering the interior storage portion.

In some embodiments, the portable imaging system further includes a cooling unit communicatively coupled to the controller and configured to reduce an internal temperature of the case.

In some embodiments, the first portion is hingedly coupled to the second portion.

In some embodiments, the portable imaging system further includes a sterilizing device configured to sterilize one or more of the case, the wireless imaging system, the imaging scope, the controller, and the display screen.

Another embodiment of the present disclosure provides a portable medical imaging system having a protective case having an external shell comprised of at least hard plastic and a first portion hingedly coupled to a second portion, the second portion including an interior storage portion comprised of material configured to deform to attenuate force, a wireless imaging system having a housing, a camera sensor disposed within the housing, a rechargeable battery, and a light source, an imaging scope removably coupled to the wireless imaging system, a controller disposed within the protective case and wirelessly coupled to the wireless imaging system, a display screen disposed on an interior surface of the first portion of the protective case, the display screen communicatively coupled to the controller and an input device, and an internal battery disposed within the protective case and coupled to one or more of the controller, the display screen, and the input device. The interior storage portion may include a plurality of recesses configured to store the wireless imaging system, the imaging scope, and the controller.

Another embodiment of the present disclosure provides a method of using a portable medical imaging system. The method includes transporting the portable medical imaging system to a first location, the portable medical imaging system including a protective case, opening the protective case to access an interior storage portion within the protective case, removing a wireless imaging system and a medical imaging scope from the interior storage portion, and transporting the portable medical imaging system to a second location, wherein the second location is remote to the first location.

The method further includes coupling the medical imaging scope to the wireless imaging system and inserting the medical imaging scope into an incision disposed on a patient proximate an anatomical region.

The method further includes viewing the anatomical region of the patient on a display screen disposed within the protective case.

The method further includes adding a sterilizing cover to one or more of the medical imaging scope, the wireless imaging system, and a battery coupled to the wireless imaging system prior to inserting the medical imaging scope into the incision.

The method further includes decoupling the medical imaging scope from the wireless imaging system and placing the medical imaging scope into a first recess disposed in the interior storage portion and placing the wireless imaging system into a second recess disposed in the interior storage portion, wherein the first recess and the second recess are separated by a material configured to deform to attenuate force.

### BRIEF DESCRIPTION OF THE DRAWINGS

The following detailed description of embodiments of the portable medical imaging system, will be better understood when read in conjunction with the appended drawings of exemplary embodiments. It should be understood, however, that the invention is not limited to the precise arrangements and instrumentalities shown.

In the drawings:

FIG. 1 is a front perspective view of a portable medical imaging system in accordance with an exemplary embodiment of the present disclosure shown in the open and powered off configuration;

FIG. 2 is a front perspective view of the portable medical imaging system of FIG. 1 shown in a powered on state;

FIG. 3A is a top planar view of the portable medical imaging system of FIG. 1;

FIG. 3B is a top planar view of the portable medical imaging system of FIG. 1 shown in the closed configuration;

FIG. 4 is a front perspective view of the portable medical imaging of FIG. 1 shown with a top portion of the protective divider removed;

FIG. 5 is a top planar view of the portable medical imaging system of FIG. 4;

FIG. 6 is a block diagram of an exemplary imaging system of the portable medical imaging system of FIG. 1;

FIG. 7 is a block diagram of the internal components of the portable medical imaging system of FIG. 1; and

FIG. 8 is a flowchart of an exemplary method of use of the portable medical imaging system of FIG. 1.

### DETAILED DESCRIPTION

Medical imaging systems are routinely used to assist in the diagnosis and treatment of patients. For example, medical imaging systems may include imaging devices that are inserted into patients to view anatomical regions of the patient on display screens to diagnose and treat different injuries, diseases, illnesses, and ailments. Traditional medical imaging systems are not self-contained systems and require many accessories and cables for operation. For example, traditional medical imaging systems require bulky displays, imaging devices, and power sources, which are all coupled to each other using many cables. This can result in many trip hazards, in addition to requiring significant room. Further, these traditional medical imaging systems require bulky equipment and are not easily transportable or portable.

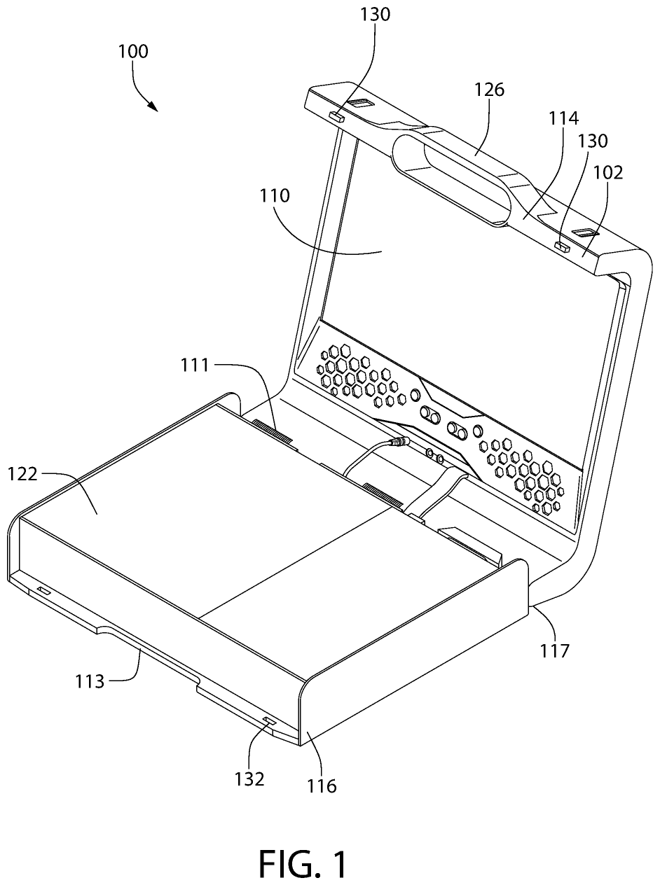

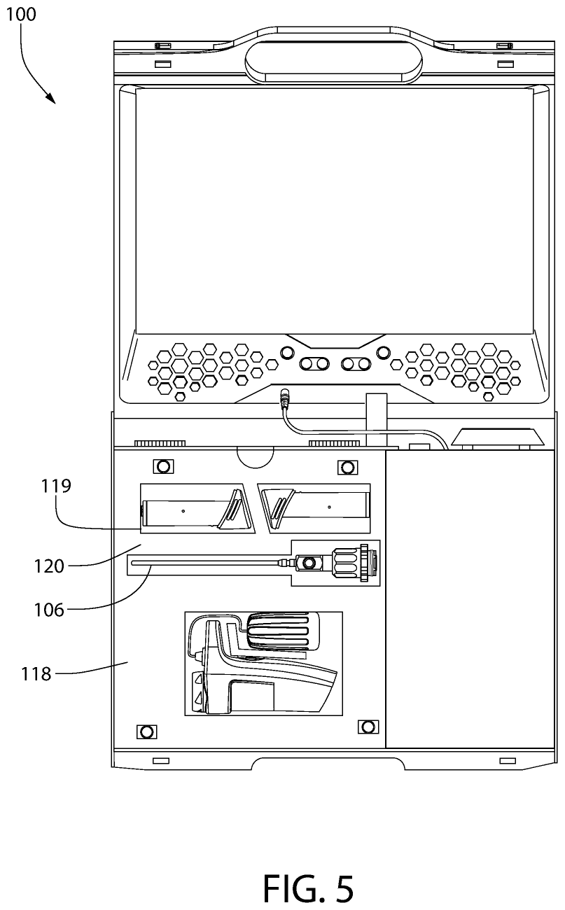

Exemplary embodiments of the present invention provide a portable medical imaging system (“portable imaging system”). Referring to FIGS. 1-7, there is shown a portable imaging system, generally designated 100. Portable imaging system 100 may be a self-contained imaging system configured to allow a user to diagnose and treat a patient. Portable imaging system 100 may be a self-contained portable medical imaging system. For example, portable imaging system 100 may include a medical imaging scope, such as an endoscope, that may be coupled to a wireless camera system to allow a user to view an anatomical region within a patient. In some embodiments, portable imaging system 100 includes a case with a built-in display screen and an imaging device stored within to allow a user to view a target area within a patient for treatment and diagnosis. The imaging device may be a wireless imaging device, such as a wireless medical imaging device. The case of portable imaging system 100 may be configured to store the imaging device and other accessories, such as batteries, cables, cords, and/or attachments for the imaging device.

In some embodiments, portable imaging system 100 includes a plurality of surgical tools configured to assist with the treatment of a patient. Portable imaging system 100 may be a surgical grade system including surgical and imaging tools for diagnosing and treating a patient. Portable imaging system 100 may be a modular system that allows a user to easily swap out and replace components, such as surgical tools and imaging tools/devices, based on the desired need. The modular configuration of portable imaging system 100 may allow for a one size fits all system. For example, a user may prefer certain types or brands of surgical tools than another user. Portable imaging system 100 being modular may allow a user to quickly swap out tools to include the tools that they desire.

Portable imaging system 100 may include a rugged case for storing one or more imaging devices and accessories within. For example, the case of portable imaging system 100 may include a built-in display screen configured to communicate with and receive images and/or video from an imaging device. When not in use, the imaging device may be securely stored within the case of portable imaging system 100 such that the imaging device can be safely transported. During use, the imaging device may be configured to communicate with one or more components disposed within the case, such as the display screen. In some embodiments, the imaging device is a wireless medical imaging device used for the treatment of diagnosis of a patient.

In some embodiments, portable imaging system 100 is self-contained within a hard, rugged case that provides protection to the components of portable imaging system 100. For example, the case of portable imaging system 100 may be configured to withstand large amounts of force (e.g., ballistic or explosive forces) without breaking or cracking. Further, the interior of portable imaging system 100 may include force attenuating materials to provide protection to the components of portable imaging system 100 when the case is subjected to large forces. In some embodiments, the hard, rugged case of portable imaging system 100 is bulletproof to allow portable imaging system 100 to be used in warzones or dangerous situations.

Portable imaging system 100 may be a portable self-contained medical imaging system that allows a user to diagnose and treat a patient anywhere without the need for a hospital or operating room. Portable imaging system 100 may allow for minimally invasive procedures in any location to treat and diagnose a patient. Portable imaging system 100 may also be used for training purposes in medical school, residencies, and fellowships. For example, portable imaging system 100 may allow a student or trainee to receive hands-on training and develop the necessary muscle memory without being restricted to a medical setting. In some embodiments, portable imaging system 100 is configured to be self-contained such that it can be used in remote locations that are far from cellular or WiFi signals. For example, portable imaging system 100 may be used in remote locations where cellular and/or WiFi signals cannot be obtained.

Portable imaging system 100 may be used in harsh or austere environments such as warzones, natural disasters, submarines, ships, aircraft, military bases, harsh environments, dangerous situations, or any other location desired. In some embodiments, portable imaging system 100 is configured for telemedicine use. For example, a user may utilize portable imaging system 100 to view a desired anatomical region within a patient, such as the stomach, and the image/video data may be sent to a remote location for viewing by a medical expert for diagnosis and/or treatment options. This allows for access to highly trained medical experts without requiring the medical expert to be in the dangerous setting or proximate the patient. Further, the portable nature of portable imaging system 100 allows for quick medical intervention to patients in austere and harsh environments, where time is a critical factor.

The portability and wireless nature of portable imaging system 100 allows for portable imaging system 100 to be used anywhere without the need for a medical setting. Portable imaging system 100 may require minimal movement of the patient. Since portable imaging system 100 can be used anywhere and does not require a hospital setting, a patient may receive medical care without having to be transported to another location. In some embodiments, portable imaging system 100 is used further away from hospitals such as in/out-patient surgical centers, physician offices, nursing homes/long term care facilities, and mobile clinics and surgical centers that can go to a patient directly. For example, a bed-bound patient at a long-term facility who is suffering from an injury could be treated at that facility and rapidly be provided rehab services instead of spending additional time at a hospital operating room or having to suffer with difficult transportation. Further, portable imaging system 100 may allow for the creation of mobile diagnostic and surgical suites that could provide minimally invasive surgery near the homes of their patients thereby decreasing the pre- and post-surgical time and allowing the patient to return to a comfortable environment.

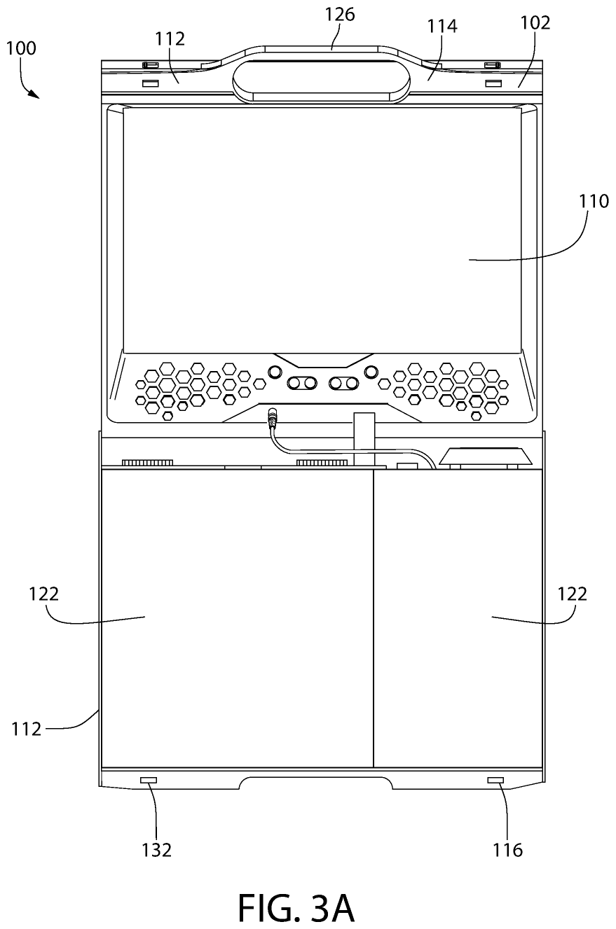

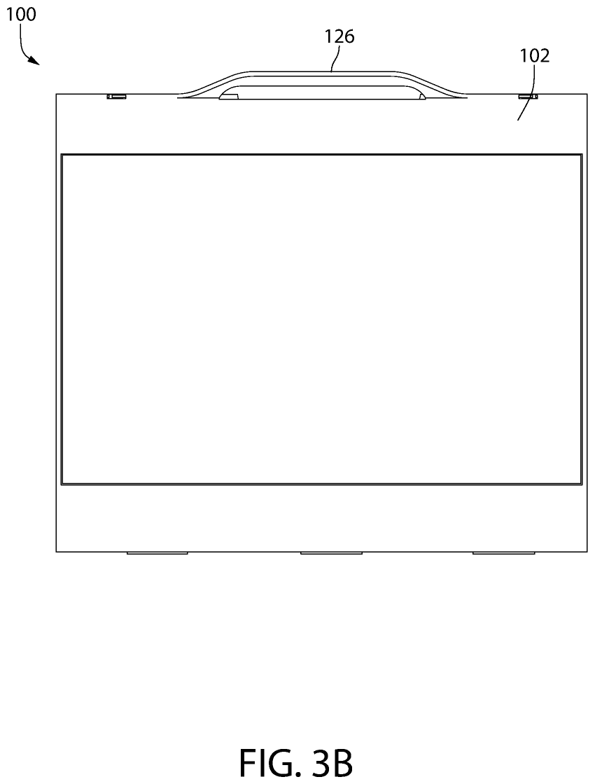

Referring to FIGS. 3A-4 and 7, portable imaging system 100 may include a housing, container, or case 102, which may be configured to store various components of portable imaging system 100. For example, portable imaging system 100 may also include imaging device 104, controller 108, display screen 110, imaging scope 106, input device 124, and internal battery 134, each of which are stored and secured within case 102. Case 102 may be configured to store and protect each of these components and may allow a user to easily retrieve one or more of these components. Case 102 may have an open configuration (FIG. 3A) and a closed configuration (FIG. 3B). In the open configuration, the interior of case 102 may be accessible. In contrast, in the closed position, a user may be prevented from accessing the contents stored within case 102. In some embodiments, case 102 includes handle 126, which allows for easy transportation and portability of portable imaging system 100. Case 102 may include one handle or multiple handles. For example, case 102 may include two, three, four, or greater than four handles. In some embodiments, case 102 is a backpack type carrying case. Case 102 may include one or more wheels to allow for easy transportation and portability of portable imaging system 100. In some embodiments, case 102 is configured to be transported using a drone. For example, case 102 may include one or more attachment points configured to be coupled to a drone or other unmanned vehicle to transport portable imaging system 100 through harsh environments and/or to remote locations.

Case 102 may include external shell 112, first portion 114, and second portion 116. Shell 112 may be configured to extend around the exterior of case 102 to provide protection to case 102. In some embodiments, shell 112 is configured to extend from first portion 114 to second portion 116 thereby providing protection for first portion 114 and second portion 116. Case 102 may be sized and shaped to allow for easy transportation and portability. In some embodiments, dimension of case 102 are 17 in×17 in×4.5 in. However, case 102 may have a length of 5 in to 30 in, 10 in to 20 in, greater than 20 in, or less than 5 in, a width of 5 in to 30 in, 10 in to 20 in, greater than 20 in, or less than 5 in, and a height of 1 in to 15 in, 5 in to 10 in, greater than 15 in, or less than 1 in. The size and shape of case 102 may vary based on the application or intended location of use of portable imaging system 100. For example, compared to conventional medical imaging systems, case 102 may be smaller and lighter to be used in warzone situations where medical evacuations are common. Case 102 may be configured to protect the components stored within case 102. In some embodiments, case 102 is substantially rectangular to allow for easy storage. For example, multiple portable imaging systems 100 may be stored in a single location and may be stacked. However, case 102 may be square, circular, trapezoidal, or any other shape desired.

Case 102 may include shell 112, which may be configured to protect case 102 and the components stored within case 102. In some embodiments, shell 112 is comprised of a hard plastic. However, shell 112 may be comprised of metal, a metal alloy, polymers, or any combination thereof. Shell 112 may be configured to withstand a large force without damage to itself or internal components stored within. In some embodiments, shell 112 is bulletproof and is configured to protect the contents stored within case 102. In some embodiments, shell 112 is waterproof to prevent water from entering the interior of case 102. Shell 112 may be comprised of a gripping material to allow for easy carrying and retrieval of case 102.

In some embodiments, shell 112 is comprised of a material configured to provide impact attenuation to case 102. For example, shell 112 may be comprised of thermoplastic polyurethane (TPU), polycarbonate, acrylonitrile-butadiene-styrene (ABS), or a similar material. In some embodiments, shell 112 is at least partially comprised of rubber or similar material and also includes a layer of Kevlar, ceramic armor, or another bulletproof material. Shell 112 may be comprised of a lightweight but hard plastic or rubber to allow for portability and transportation of portable imaging system 100. In some embodiments, shell 112 is comprised of a lightweight material, while still providing protection to the interior of case 102 allowing case 102 to be easily carried while also providing protection for the components stored within.

Case 102 may include first portion 114 and second portion 116. In some embodiments, first portion 114 is coupled to second portion 116 to form case 102. First portion 114 may be hingedly or pivotally coupled to second portion 116 such that first portion 114 pivots relative to second portion 116. In some embodiments, second portion 116 includes first end 111 and second end 113. First portion 114 may be hingedly coupled to second portion 116 at first end 111 and removably coupled to second portion 116 at second end 113. In some embodiments, first end 111 is opposite from second end 113.

In some embodiments, case 102 has a closed configuration and an open configuration. Case 102 may be in the closed configuration (FIG. 3B) when first portion 114 is secured to second portion 116 at second end 113 and case 102 may be in the open configuration when first portion 114 is decoupled from second portion 116 at second end 113. For example, case 102 may be opened by decoupling first portion 114 from second portion 116 at second end 113 and hinging or pivoting first portion 114 relative to second portion 116 at first end 111 resulting in first portion 114 moving away from second end 113. In the open configuration, first portion 114 may remain coupled to second portion 116 at first end 111. However, in some embodiments, first portion 114 may be decoupled from second portion 116 at both first end 111 and second end 113 to access the interior of case 102. In some embodiments, first portion 114 is configured to rotate or pivot up to 135° relative to second portion 116. However, first portion 114 may be configured to pivot up to 90°, 120°, 180°, 270°, or 360° relative to second portion 116. For example, first portion 114 may be configured to 360° relative to second portion 114 such that shell 112 of first portion 114 is proximate or abuts shell 112 of second portion 116.

Shell 112 may cover one or both of first portion 114 and second portion 116. In some embodiments, shell 112 covers at least a portion of first portion 114 and/or second portion 116. For example, shell 112 may cover the exterior surface of first portion 114 and/or the exterior surface of second portion 116. Shell 112 may cover at least a portion of both first portion 114 and second portion 116 and may include divider 117 where first portion 114 is coupled to second portion 116 to allow shell 112 to hinge and pivot at the intersection of first portion 114 and second portion 116. Divider 117 may allow shell 112 to hinge and pivot to allow first portion 114 and second portion 116 to move relative to each other. In some embodiments, shell 112 is bifurcated such that a portion of shell 112 covers at least a portion of first portion 114 and another portion of shell 112 covers at least a portion of second portion 116.

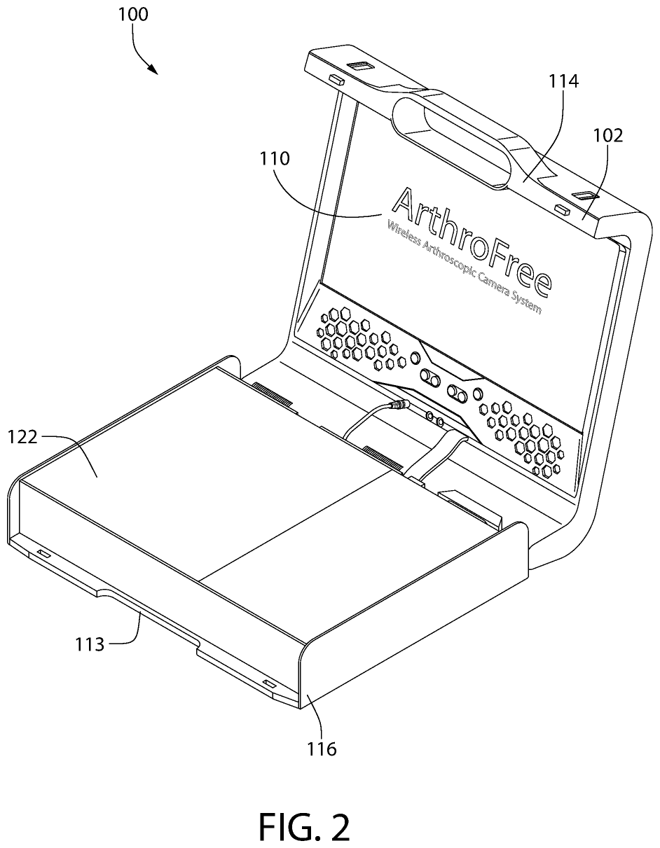

Referring to FIGS. 1 and 2, first portion 114 may be secured to second portion 116 via locking mechanism 130 and locking recess 132. First portion 114 may include locking mechanism 130 and second portion 116 may include locking recess 132, which may be configured to receive locking mechanism 130. First portion 114 may include one or more locking mechanisms 130 and second portion 116 may include an equivalent number of locking recesses 132. In some embodiments, second portion 116 includes locking mechanism 130 and first portion 114 includes locking recess 132. Locking recess 132 may receive and secure locking mechanism 130 to secure and lock first portion 114 to second portion 116 thereby locking and securing case 102 in the closed configuration. First portion 114 may be secured and locked to second portion 116 at one or both of first end 111 and second end 113. In some embodiments, case 102 includes a sliding lever configured to push locking mechanism 130 out of locking recess to decouple first portion 114 from second portion 116 and allowing access to the interior of case 102. In some embodiments, case 102 may include a different style of locking or latching mechanisms. For example, case 102 may include magnets, fasteners, buttons, latches, electronic locks, biometric locks, fingerprint locks, retinal locks, speech activated locks, or any other type of locking mechanism to secure and lock case 102.

Portable imaging system 100 may include display screen 110. Display screen 110 may be a liquid crystal display (LCD) panel, a light emitting diode (LED) panel, an organic light emitting diode (OLED) panel, quantum dot light emitting diode (QLED) panel, or other type of display panel. Display screen 110 may have a resolution of up to 7680×4320, 4096×2160, 3840×2160, 2048×1152, 1920×1080, 1280×720, or 640×480, or any other resolution in between or greater than 7680×4320. Display screen 110 may have a refresh rate of up to 30 Hz, 50 Hz, 60 Hz, 120 Hz, 240 Hz, 480 Hz, or any other refresh rate in between or higher than 480 Hz. Display screen 110 may include an anti-reflective or anti-glare coating or layer to reduce or prevent glare.

In some embodiments, display screen 110 is disposed on first portion 114. For example, display screen 110 may be disposed on an interior surface of first portion 114 such that when case 102 is in the closed configuration, display screen 110 is proximate second portion 116. Display screen 110 may have an off configuration (FIG. 1) and an on configuration (FIG. 2). Display screen 110 may be configured to provide bright and accurate colors at a high resolution regardless of the viewing angle. In some embodiments, display screen 110 is a durable panel configured to provide high resolution while also being able to sustain impact.

In some embodiments, display screen 110 may be removably coupled to case 102. For example, display screen 110 may be removably coupled to an interior surface of first potion 114. Display screen 110 may be wirelessly coupled to controller 108 such that display screen 110 can be removed from case 102 and used remote to case 102. In some embodiments, display screen 110 is a portable mobile device (e.g., a tablet). Display screen 110 may be a mobile device having a touchscreen. In some embodiments, display screen 110 is removable from case 102 and wirelessly coupled to controller 108 and a remote server. For example, display screen 110 may be transportable relative to case 102 and allow a user to view image and/or video data received by controller 108 from imaging device 104.

Display screen 110 may be further configured to receive data from a remote server. In some embodiments, display screen 110 is configured to receive image and/or video data from imaging device 104 to a remote server while simultaneously transmitting and receiving data from a remote server. For example, a user of portable imaging system 100 may decouple display screen 110 from case 102 to place it closer to the patient or use it in a more ergonomical or comfortable position. Display screen 110 may receive image and/or video data from imaging device 104 and transmit the image and/or video data to a remote server or remote party. Simultaneously, display screen 110 may receive data from a remote server to allow the user to video chat or audio chat with a remote individual that is also viewing the image and/or video data from imaging device 104. This allows a user of portable imaging system 100 to wirelessly receive data from imaging device 104 while, for example, communicating with a remote individual via video conferencing to assist with diagnosis or treatment of the patient. The remote user may be a medical expert in fielding relating to the diagnosis and/or treatment of the patient for which portable imaging system 100 is being used.

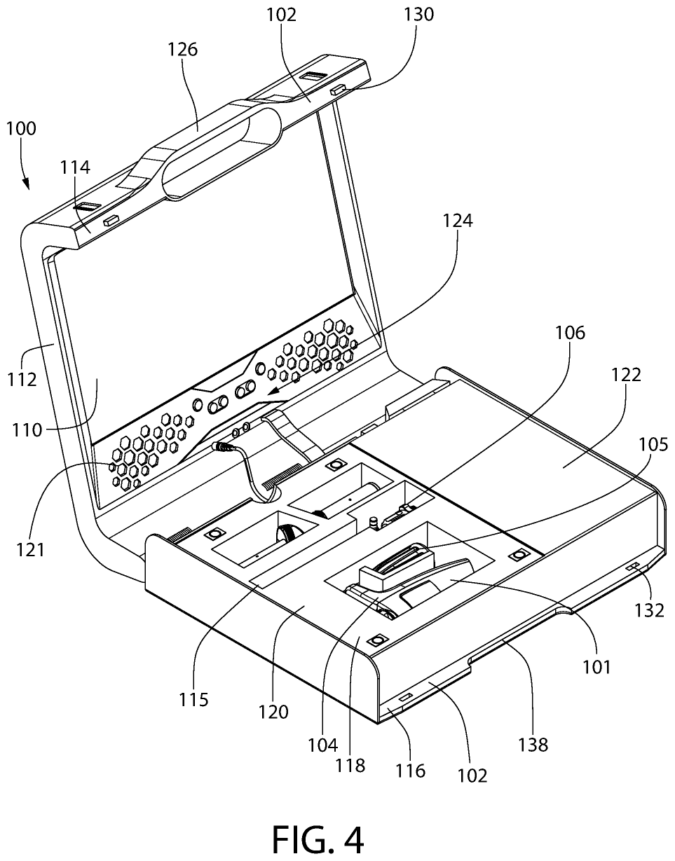

Referring to FIGS. 4-7, second portion 116 may include interior storage portion 118. Storage portion 118 may be configured to receive components stored within case 102 of portable imaging system 100. For example, storage portion 118 may include one or more recesses 119 configured to receive and secure various components (e.g., surgical tools, medical equipment, imaging devices, accessories). In some embodiments, recesses 119 are each sized and shaped based on the component stored and secured within. For example, recesses 119 may have varying heights, widths, and lengths depending on the size of the component stored within. Multiple recesses 119 may be disposed within storage portion 118 such that one recesses 119 is proximate another recess 119. In some embodiments, when case 102 is in the closed configuration, first portion 114 covers recesses 119 to prevent the components stored within recesses 119 from exiting or falling out of its respective recess 119. This ensures that during transportation and movement of portable imaging system 100 items do not fall out of recesses 119 and become damaged.

Storage portion 118 may include force attenuating material 120 (“material 120”). Material 120 may be comprised of polyurethane, polystyrene, polypropylene, foam, rubber, silicone, polymers (e.g., viscoelastic polymers), or any combinations thereof or other type of material configured to absorb and/or attenuate force. In some embodiments, material 120 is configured to deform to absorb, attenuate, and/or dissipate force applied to storage portion 118 and/or case 102. Each recess 119 may be formed within material 120 such that material 120 provides protection to the component or item stored within recess 119. Material 120 may be configured to protect the components stored in recesses 119 and may assist in attenuating impact force delivered to case 102 and portable imaging system 100. In some embodiments, recesses 119 are cutouts of material 120.

Referring to FIGS. 1-4, portable imaging system 100 may include protective divider 122. Divider 122 may be disposed within the interior of case 102 and may be configured to protect the components stored within the interior of case 102, such as within recesses 119 of storage portion 118. In some embodiments, divider 122 is comprised of a hard plastic. Divider 122 may include a soft material on the side facing the storage portion 118 to prevent damage to the components stored within storage portion 118. Divider 122 may be comprised of one or more pieces. For example, divider 122 may comprised of two pieces to allow for partial access to some components stored within interior storage portion 118, while still protecting the other components. However, divider 122 may be comprised of three, four, five, six, or more than six pieces. In some embodiments, to access the components stored within storage portion 118, a user has to remove one or more dividers 122 (FIG. 4). When case 102 is in the closed position, divider 122 may be disposed between first portion 114 and second portion 116. When case 102 is in the closed position, divider 122 may be configured to prevent components within recesses 119 from falling out and/or damaging the interior of first portion 114. For example, first portion 114 may include display screen 110 disposed on the interior of first portion 114 and divider 122 may be disposed between display screen 110 and storage portion 118 when case 102 is in the closed position to prevent the components within recesses 119 of storage portion 118 from contacting and/or damaging display screen 110 during movement and transportation of case 102.

Referring to FIGS. 4-7, portable imaging system 100 may include imaging device 104. Imaging device 104 may be stored within case 102 when not being used by a user. For example, imaging device 104 may be stored within case 102 until a user elects to use imaging device 104 for treatment and/or diagnosis of a patient. In some embodiments, imaging device 104 is a wireless handheld imaging device, which allows for portability and increased maneuverability of imaging device 104. In some embodiments, imaging device 104 includes housing 101, camera sensor 103, light source 105, and camera battery 107. Imaging device 104 may be similar to the wireless imaging system of U.S. Pat. Nos. 10,610,089 and 10,932,658, and U.S. Patent Application Publication No. 2019/0167074, which are hereby incorporated by reference in their entirety.

Imaging device 104 may include housing 101. Housing 101 may comprise the external surface of imaging device 104. In some embodiments, housing 101 is comprised of a durable material to allow for more rugged use of imaging device 104. For example, housing 101 may be comprised of a durable polymer or metal to allow imaging device 104 to be dropped or receive one or more impacts without damaging imaging unit 104. Housing 101 may be made via molding, casting, reductive processes, and/or 3D printing.

Housing 101 may be sized and shaped to be handheld to allow for easy holding and maneuvering of imaging device 104 by a user. For example, imaging device 104 may be wireless and operated by a single hand to allow for maneuverability and also allow the user to use their other hand for other purposes (e.g., using a surgical tool, holding open an incision on the patient, providing irrigation to and into the incision, adjusting settings on display screen 110). Camera sensor 103 may be disposed within housing 101 and may be configured to receive and transmit image data. For example, imaging device 104 may be communicatively coupled to a controller (e.g., controller 108), and camera sensor 103 may transmit image data to the controller. Imaging device 104 may be configured to transmit images/videos to a display screen, such as the display screen disposed on first portion 114 (e.g., display screen 110).

In some embodiments, imaging device 104 is configured to perform image processing on the images acquired by camera sensor 103. Imaging device 104 may also include data storage for storing image data acquired by camera sensor 103. In some embodiments, the data storage is coupled to controller 108 and is configured to receive and transmit the image data from and to controller 108. In some embodiments, controller 108 includes the data storage. For example, the data storage may be disposed within controller 108.

Imaging device 104 may include light source 105. Light source 105 may be disposed within housing 101 or may be coupled to housing 101. In some embodiments, light source 105 is coupled to housing 101 via a cable. Light source 105 may be similar to the light source of U.S. Pat. Nos. 10,488,018, which is hereby incorporated by reference in its entirety. Light source 105 may be coupled to housing 101 to provide light to imaging device 104. In some embodiments, light source 105 is configured to illuminate a target area during use of imaging device 104. Light source 105 may be configured to generate and output light. In some embodiments, light source 105 includes an illumination source. The illumination source may be a laser or a light emitting diode.

In some embodiments, light source 105 is configured convert light from the illumination source to an output light. The output light may have a different peak wavelength than the light from the illumination source. In some embodiments, light from the illumination source has a peak wavelength light of 440 nm to 470 nm and the output light from light source 105 has a peak wavelength of 480 nm to 770 nm. In some embodiments, light source 105 includes a converter configured to convert light from the illumination source to output light having a different wavelength. The converter may include phosphor particles volumetrically disposed within a substrate. In some embodiments, the substrate may be a homogenous composite substrate of non-converting material (e.g., plastic, acrylic, glass, ceramic). The phosphor particles being volumetrically disposed within the converter of light source 105 allows the converter to more efficiently convert light from the illumination source to the output light. Light source 105 may include one or more optical elements configured to condition, collimate, filter, direct, and/or focus the light from the illumination source and/or the output light.

In some embodiments, imaging device 104 also includes battery 107. In some embodiments, battery 107 is disposed within housing 101. However, battery 107 may be coupled to housing 101. In some embodiments, battery 107 is removably coupled to housing 101 such that battery 107 can be easily replaced. Battery 107 may be a rechargeable battery. For example, battery 107 may be a rechargeable battery that is configured to power imaging device 104 for 30 minutes, 45 minutes, one hour, one hour fifteen minutes, one hour and thirty minutes, two hours, or greater than two hours. Battery 107 may be configured to be recharged by an internal battery disposed within case 102. For example, case 102 may include internal battery 134 which may be configured to provide power and/or charge to various devices or components, such as battery 107. In some embodiments, portable imaging system 100 includes a plurality of batteries 107 such that during use of imaging device 104, when one battery is depleted, another battery may be quickly coupled to imaging device 104 with minimal interruption of imaging device 104. In some embodiments, battery 107 is a disposable battery. Battery 107 may be configured to provide power to camera sensor 103 and light source 105 of imaging device 104. Battery 107 may be easily cleaned and may be sterilizable to allow for repeated use without causing contamination to portable imaging system 100.

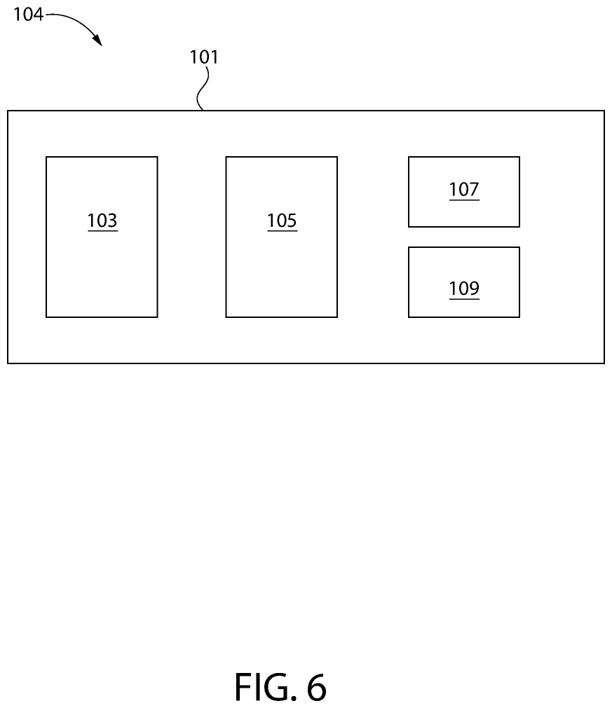

Referring to FIG. 6, imaging device 104 may include wireless communication chip or wireless communication system 109. Wireless communication chip 109 may be disposed within housing 101 and may be coupled to camera sensor 103. Wireless communication chip 109 may receive image data from camera sensor 103 and/or a data storage device and may wirelessly transmit the image data to a controller disposed within case (e.g., controller 108) or a remote source (e.g., a remote server). Wireless communication chip 109 may include various wireless modalities such as Bluetooth, WiFi, Ultrawideband (UWB), near field communication (NFC), or radio frequency (RF).

Referring to FIGS. 4-6, portable imaging system 100 may include imaging scope 106. Imaging scope 106 may be a medical imaging scope configured to be inserted into a living organism, such as a human or animal, or a cadaver model. For example, imaging scope 106 may be as a gastroscope, a bronchoscope, a cystoscope, a ureteroscope, an arthroscope, a colonoscope, or any other type of medical imaging scope. Imaging scope 106 may be rigid or flexible and may be configured to enter a patient's body to treat or diagnose an anatomical region of the patient. For example, imaging scope 106 may be a gastroscope to diagnose and/or treat the stomach of a patient. In some embodiments, imaging scope 106 is configured to be single use. For example, imaging scope 106 may be a disposable endoscope used only once for a single patient. However, imaging scope 106 may be a multi-use medical imaging scope that may be sterilized between uses. In some embodiments, portable imaging system 100 includes a plurality of imaging scopes 106. For example, case 102 may be configured to store multiple imaging scopes 106 to allow for multiple uses of imaging device 104. For example, portable imaging system 100 may be taken to a remote location with several injured patients and imaging device 104 may be used with multiple imaging scopes 106, one for each patient, to treat and diagnosis each patient.

In some embodiments, imaging scope 106 includes cover 115. Cover 115 may be a sterilizing cover that receives a portion or the entirety of imaging scope 106, imaging device 104, and/or camera battery 107. In some embodiments, cover 115 protects imaging scope 106 from dust and debris prior to imaging scope 106 being inserted into the patient. Portable imaging system 100 may include a plurality of covers 115. For example, case 102 may be configured to store and secure multiple covers 115, such as covers 115 of different sizes and shapes. In practice, cover 115 may be removed from imaging scope 106 prior to insertion of imaging scope 106 into the patient.

In some embodiments, cover 115 includes a sterilizing device. For example, cover 115 may include a built-in UV-light sterilizer configured to sterilize imaging scope 106 prior to each use. Cover 115 may allow a single imaging scope 106 to be used multiple times. In some embodiments, the sterilizing device is separate from cover 115 and is disposed within case 102. For example, the sterilizing device may be stored within case 102 and may be used by the user to sterilize imaging scope 106 prior to use. Prior to use of imaging scope 106, a user may remove cover 115 and then may use the sterilizing device to ensure that imaging scope 106 is sterile. In some embodiments, sterilizing device is a UV-light sterilizer. However, sterilizing device may be an alcohol or iodine-based device for cleaning and sterilizing to imaging scope 106.

In some embodiments, cover 115 is configured to cover a portion or the entirety of imaging device 104. For example, cover 115 may extend from imaging scope 106 to imaging device 104 such that cover 115 covers both imaging scope 106 and imaging device 104. In some embodiments, imaging device 104 includes battery 107 and cover 115 is configured to cover imaging scope 106, imaging device 104, and battery 107. In practice, certain environments may not allow for quick sterilization of imaging scope 106 and/or imaging device 104. Therefore, cover 115 may be used instead of sterilizing imaging scope 106 and/or imaging device 104.

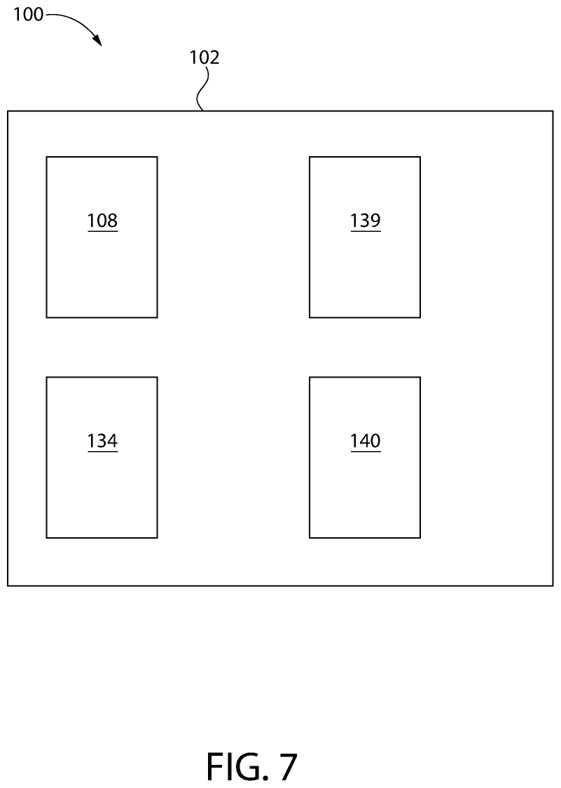

Referring to FIGS. 4 and 7, portable imaging system 100 may include controller 108. Controller 108 may be a processor, microcontroller, or logic controller. Controller 108 may be disposed within second portion 116 of case 102. However, controller 108 may be disposed within first portion 114. In some embodiments, controller 108 is communicatively coupled to imaging device 104. For example, controller 108 may be configured to wirelessly receive image data from imaging device 104. Controller 108 may be coupled to internal battery 134 stored within case 102 and internal battery 134 may be configured to provide power to controller 108. However, controller 108 may be plugged into a wall, external power source, or another power supply (e.g., solar panels). In some embodiments, controller 108 receives data from additional sources. For example, controller 108 may receive data from various components such as imaging device 104, battery 107, internal battery 134, display screen 110, cooling unit 139, and/or input device 124. In some embodiments, battery 107 and/or internal battery 134 send battery life information to controller 108.

Imaging device 104 may transmit image data and/or diagnostic information to controller 108. In some embodiments, controller 108 receives data from imaging device 104 and transmits the data to display screen 110 for display. For example, controller 108 may receive image data from imaging device 104 in real-time, process the image data in real-time, and transmit the processed image data to display screen 110 for live viewing by the user. Controller 108 may process image data in real-time to allow the user to view a live image of the visualization site of imaging device 104. Display screen 110 may be communicatively coupled to controller 108. For example, controller 108 may transmit image data that it receives from imaging device 104 to display screen 110. In some embodiments, display screen 110 displays live images/videos from imaging device 104 via controller 108. In some embodiments, display screen 110 is a touch screen configured to receive inputs and commands from a user. Display screen 110 may provide status and updates of various components of portable imaging system 100. For example, display screen 110 may be commutatively coupled to controller 108, imaging device 104, battery 107, and/or internal battery 134 and display screen 110 may be configured to display the status of each device. In some embodiments, display screen 110 allows a user to control the brightness, zoom level, contrast, or other visual properties of images/videos transmitted by imaging device 104.

In some embodiments, portable imaging system 100 includes additional components that transmit data to controller 108. For example, portable imaging system 100 may include a global positioning system (GPS) chip, a Galileo chip, a GLONASS chip, or other positioning modality that transmits location data to controller 108. In some embodiments, portable imaging system 100 includes storage device 140 and controller 108 is communicatively coupled to storage device 140. Storage device 140 may be disposed within case 102 or may be external to case 102. In some embodiments, storage device 140 is a removable storage coupled to case 102 and commutatively coupled to controller 108. Controller 108 may receive data from storage device 140 and may transmit data to storage device 140 for storage. In some embodiments, portable imaging system 100 is configured to encrypt the data stored on storage device 140 to prevent inadvertent disclosure of protected health information.

Controller 108 may be configured to receive and transmit information to a remote location. In some embodiments, controller 108 performs the image processing for image data received from imaging device 104 prior to displaying the image data on display screen 110. Case 102 may include input device 124 configured to control various components of portable imaging system 100, such as display screen 110. Controller 108 may receive data from input device 124 and transmit data to display screen 110. For example, a user may use input device 124 to increase the brightness of the display screen 110. The data associated with the user selecting an input of input device 124 is transmitted from input device 124 to controller 108, which is transmitted to display screen 110 thereby increasing the brightness of display screen 110.

In some embodiments, display screen 110 is communicatively coupled to input device 124. Input device 124 may receive input from a user and transmit data to display screen 110 via controller 108. Input device 124 may be a keyboard, a plurality of buttons, a touchscreen, or a remotely located device such as a tablet or a smartphone. In some embodiments, input device 124 is a mobile device (e.g., a tablet or smartphone) communicatively coupled to portable imaging system 100. In some embodiments, display screen 110 is a touch screen and receives inputs from a user. Input device 124 may be configured to receive commands from a user, which may be transmitted to controller 108. In some embodiments, case 102 includes speakers 121. Speakers 121 may be configured to provide audio output to a user such as instructions. In some embodiments, portable imaging system 100 allows for communication with a remote individual or expert. Display screen 110 may be provide visual instructions provided by a remote individual and/or speakers 121 may provide audio instructions from the remote individual.

In some embodiments, controller 108 is configured to stream image data from imaging device 104 to a remote site. For example, controller 108 may be configured to stream live images received from imaging device 104 to a remote viewing system via a mobile connection (e.g., 5G), a satellite connection, a wireless connection (e.g., WiFi) or via a wired connection. In some embodiments, controller 108 is configured to stream live images on display screen 110 and a remote viewing system (e.g., second display or remote display) simultaneously to allow a user and a third-party (e.g., remote medical expert) to simultaneously view the visualization site of imaging device 104.

With continued reference to FIGS. 4 and 7, portable imaging system 100 may include internal battery 134. Internal battery 134 may be coupled to one or more of controller 108, input device 124, display screen 110, cooling unit 139, battery 107, imaging device 104, cover 115, sterilization device and/or storage device 140. In some embodiments, internal battery 134 can be coupled to imaging device 104 if battery 107 is depleted or malfunctioning. Internal battery 134 may be a rechargeable battery disposed within case 102. In some embodiments, internal battery 134 is disposed within second portion 116 between shell 112 and interior storage portion 118. However, internal battery 134 may be disposed within interior storage portion 118. However, internal battery 134 may be disposed within front portion between shell 112 and display screen 110 or below display screen 110. Internal battery 134 may be removably coupled to case 102 to allow a user to easily replace internal battery 134. Internal battery 134 may include a durable exterior shell to protect internal battery 134. For example, internal battery 134 may include a bulletproof or puncture proof shell to prevent damage to internal battery 134 during use of portable imaging system 100.

In some embodiments, case 102 includes solar panels configured to charge internal battery 134 and/or battery 107 when portable imaging system 100 is used in outdoor environments. Internal battery 134 may be configured to charge battery 107 when battery 107 is disposed within case 102. For example, a user may place battery 107 within interior storage portion 118 and internal battery 134 may be coupled to battery 107 once battery 107 is inserted within interior storage portion 118. In some embodiments, internal battery 134 is configured to wirelessly charge battery 107. Internal battery 134 may be a lightweight yet powerful battery to allow for easy portability and transportation of portable imaging system 100. In some embodiments, internal battery 134 is communicatively coupled to controller 108 and is configured to transfer data associated with its power status to controller 108. For example, internal battery 134 may transmit data associated with the charge level of internal battery 134 and controller 108 may display the charge level to the user via display screen 110.

Referring to FIGS. 4-5 and 7, portable imaging system 100 may include cooling unit 139. Cooling unit 139 may be configured to cool portable imaging system 100 to prevent portable imaging system 100 from overheating. For example, due to the heat generated by one or more of controller 108, internal battery 134, display screen 110, input device 124, battery 107, and imaging device 104, portable imaging system 100 may overheat resulting in damage to portable imaging system 100, case 102, the components stored within case 102, and/or the user. In some embodiments, cooling unit 139 includes one or more fans that are powered by internal battery 134 and are configured to circulate air throughout case 102 and out through outlet 138. In some embodiments, cooling unit 139 includes a heatsink and/or a one or more heatpipes. The heatsink and heatpipes may be coupled to one or more fans that are powered by internal battery 134. However, the fans may remain decoupled from the heatsink and/or heatpipes.

In some embodiments, the fans are configured to circulate air away from the heatsink and/or heatpipes and out through outlet 138. Cooling units 139 may include thermoelectric technologies, evaporative cooling technologies, vapor chamber technologies, and other heat transfer technologies. Cooling unit 139 may be coupled to controller 108 and may reduce the internal temperature of case 102 and portable imaging system 100. In some embodiments, cooling unit 139 includes passive cooling methodology such as conduction, convection, and/or radiation to cool a component. For example, cooling unit 139 may include one or more heatsinks and/or heatpipes to assist with the passive cooling of portable imaging system 100. However, cooling unit 139 may use a combination of passive cooling and active cooling methodologies.

In some embodiments, portable imaging system 100 is configured to be modular. For example, a user may replace any component of portable imaging system 100 with another desired component without disrupting the functionality of portable imaging system 100. In some embodiments, a user may replace storage portion 118 based on the desired layout of recesses 119 or replace a large internal battery 134 with a smaller internal battery 134 to allow for easier transportability and portability. Further, portable imaging system 100 may be completely self-contained and unitary. In use, portable imaging system 100 may not require the need for any other components to diagnose and/or treat a patient.

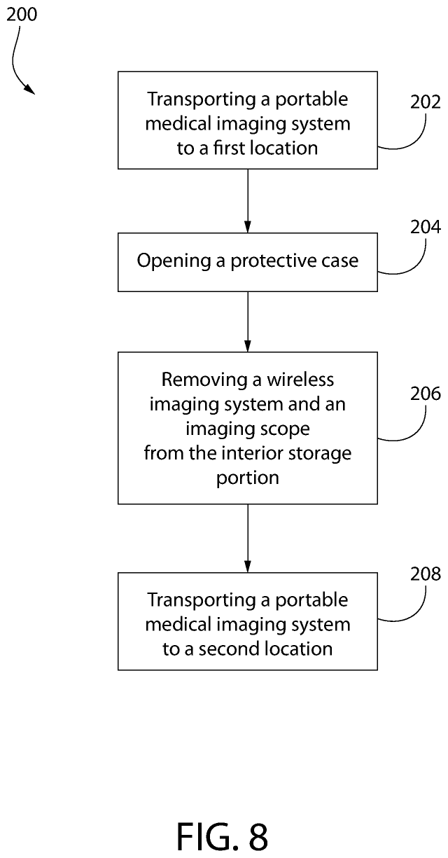

Referring to FIG. 8, a flowchart showing a method 200 of using portable imaging system 100 is shown. Method 200 may include step 202 of transporting portable imaging system 100 to a first location (e.g., a location where a first patient is located). Method 200 may include step 204 of opening case 102. Case 102 may be opened by decoupling first portion 114 from second portion 116 or second portion 116 from first portion 114. Method 200 may include step 206 of removing imaging device 104 and imaging scope 106 from interior storage portion 118 of case 102. Imaging device 104 may be a handheld wireless camera system. Imaging scope 106 may be coupled to imaging device 104. An incision may be made on the first patient and imaging scope 106 may be inserted into the incision disposed on the first patient. The incision may be disposed on the first patient proximate an anatomical region. The anatomical region may be a region of the first patient targeted for diagnosis and/or treatment. Images and videos from imaging scope 106 and imaging device 104 may be transmitted to controller 108 and display screen 110 disposed within case 102. The image and video data may be stored on storage device 140, which may be encrypted. In some embodiments, the image and video data may be wirelessly transmitted to a remote location (e.g., remote server). Additional surgical tools or imaging tools that are stored within case 102 may be used on the first patient to adequately treat and diagnosis the first patient. Upon completion of the treatment and/or diagnosis of the first patient, imaging scope 106 may then be removed from the first patient. Imaging device 104 and imaging scope 106 may then be placed within storage portion 118 of case 102 and case 102 may be powered down for transportation. During transportation, battery 107 of imaging device 104 may be charging for future use. In some embodiments, battery 107 is removed from imaging device 104 and placed in recess 119 for wirelessly charging of battery 107. In other embodiments, battery 107 is coupled to imaging device 104 and charged while remaining coupled to imaging device 104.

Method 200 may include step 208 of transporting portable imaging system 100 to a second location (e.g., a location where a second patient is located). For example, the second location may be remote from the first patient requiring the user to travel from the first patient to the second patient. In some embodiments, the first patient is not proximate the second patient. For example, the first patient may be away or distant from the second patient. Once portable imaging system 100 has arrived at the second patient, a sterilizing device and/or cover 115 may be used to sterilize imaging scope 106. In some embodiments, a new imaging scope 106 is used and coupled to imaging device 104. Battery 107 may be charged and inserted or coupled to imaging device 104 or a different fully charged battery 107 may be inserted or coupled to imaging device 104. An incision may be made within the second patient and imaging scope 106 may be inserted into the incision of the second patient to view a desired anatomical area of the second patient.

In some embodiments, the anatomical region of the patient may be viewed on display screen 110. Display screen 110 may be disposed on first portion 114 of case 102. In some embodiments, cover 115 may be removed from one or more of imaging scope 106, imaging device 104, and battery 107 prior to inserting imaging scope 106 into the incision on the patient. For example, during use, cover 115 may be removed from imaging scope 106 prior to insertion into the patient, but cover 115 may remain on camera system 100 and/or battery 107. Imaging scope 106 may be decoupled from imaging device 104 and placed into case 102. Placing imaging scope 106 into case 102 may include placing imaging scope 106 into a first recess 119 disposed in interior storage portion 118 and placing imaging device 104 into a second recess 119 disposed in interior storage portion 118. In some embodiments, the first recess and the second recess are separated by force attenuating material 120.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

1. A portable imaging system comprising:
a case having a first portion coupled to a second portion, the second portion including an interior storage portion;
a wireless imaging system having a housing, a camera sensor disposed within the housing, and a light source;
an imaging scope removably coupled to the wireless imaging system;
a controller communicatively coupled to the wireless imaging system; and
a display screen disposed on an interior surface of the first portion of the case, the display screen communicatively coupled to the controller,
wherein the interior storage portion is configured to receive and store the wireless imaging system and the imaging scope.

2. The portable imaging system of claim 1 further comprising:
a sterilizing cover configured to be disposed over one or more of the imaging scope, the wireless imaging system, and a battery coupled to the wireless imaging system.

3. The portable imaging system of claim 2 further comprising:
a sterilizing device configured to sterilize an object received by the sterilizing cover.

4. The portable imaging system of claim 1, wherein the case has an external shell comprised of one or more of a substantially hard plastic and rubber.

5. The portable imaging system of claim 4, wherein the external shell is comprised of a different material than a material of the interior storage portion.

6. The portable imaging system of claim 5, wherein the material of the interior storage portion is comprised of material configured to deform to attenuate force.

7. The portable imaging system of claim 1 further comprising:
an internal battery disposed within the case and coupled to one or more of the controller and the display screen, wherein the internal battery is configured to charge a battery coupled to the wireless imaging system.

8. The portable imaging system of claim 1, wherein the interior storage portion is comprised of material configured to dissipate force.

9. The portable imaging system of claim 1, wherein the interior storage portion includes a first recess sized and shaped to secure the wireless imaging system and a second recess sized and shaped to secure the imaging scope, the first recess being different than the second recess.

10. The portable imaging system of claim 1, wherein the case includes a locking mechanism to secure and lock the first portion to the second portion.

11. The portable imaging system of claim 1 further comprising:
a protective cover covering the interior storage portion.

12. The portable imaging system of claim 1 further comprising:
a cooling unit communicatively coupled to the controller and configured to reduce an internal temperature of the case.

13. The portable imaging system of claim 1, wherein the first portion is hingedly coupled to the second portion.

14. The portable imaging system of claim 1, wherein the camera sensor transmits one or more images to the display screen via the controller.

15. The portable imaging system of claim 1, wherein the wireless imaging system includes a rechargeable battery.

16. The portable imaging system of claim 1, wherein the controller is wirelessly coupled to the wireless imaging system.

17. The portable imaging system of claim 1 further comprising:
a sterilizing device configured to sterilize one or more of the case, the wireless imaging system, the imaging scope, the controller, and the display screen.

18. A portable medical imaging system comprising:
a protective case having an external shell comprised of at least hard plastic and a first portion hingedly coupled to a second portion, the second portion including an interior storage portion comprised of material configured to deform to attenuate force;
a wireless imaging system having a housing, a camera sensor disposed within the housing, a rechargeable battery, and a light source;
an imaging scope removably coupled to the wireless imaging system;
a controller disposed within the protective case and wirelessly coupled to the wireless imaging system;
a display screen disposed on an interior surface of the first portion of the protective case, the display screen communicatively coupled to the controller and an input device; and
an internal battery disposed within the protective case and coupled to one or more of the controller, the display screen, and the input device,
wherein the interior storage portion includes a plurality of recesses configured to store the wireless imaging system, the imaging scope, and the controller.

19-23. (canceled)
